(19) United States
(12) Patent Application Publication
(10) Pub. No.: US 2023/0023818 A1
(43) Pub. Date: Jan. 26, 2023

(54) Hepatoprotective Effects of Palauan Folk Medicine

(71) Applicant: Christopher Kitalong, Koror (PW)

(72) Inventor: Christopher Kitalong, Koror (PW)

(21) Appl. No.: 17/856,956

(22) Filed: Jul. 2, 2022

Related U.S. Application Data
(63) Continuation of application No. 16/749,849, filed on Jan. 22, 2020.

(51) Int. Cl. A61K 36/53; A61K 36/748; A61K 36/76; A61K 36/83; A61K 36/25; A61K 36/85; A61P 1/16

(52) U.S. Cl. CPC A61K 36/53; A61K 36/748; A61K 36/76; A61K 36/83; A61K 36/25; A61K 36/85; A61P 1/16

(57) ABSTRACT

The present invention relates to herbal compositions comprising the botanicals from the Palauan medicinal plants. Examples of these botanicals include but are not limited to; Astronidium palauense, Averrhoa bilimbi, Flacourtia rukam, Gmelina palawensis, Hedyotis korrorensis, Lygodium microphyllum, Manilkara udoido, Morinda pedunculata, Osmoxylon oliveri, Phaleria nisidai, or Premna obtusfolia, or extracts thereof, which are useful in treating liver diseases, particularly those with viral etiology. The compositions of the invention have demonstrated outstanding efficacy in the treatment of patients with hepatic disorders. Compositions of the present invention have also inhibited the activities of liver-damaging virus activities, such as HCV. The preferred compositions contain the botanical ingredients comprising the fatty acids or the phenolic constituents from the Palauan medicinal plants.

This application is related to and claims priority to Ser. No. 16/749,849, filed 22 Jan. 2020, entitled “HEPATOPROTECTIVE EFFECTS OF PALAUAN FOLK MEDICINE”, which application is specifically incorporated by reference herein.

### FIELD OF THE PRESENT INVENTION

The present invention relates to an herbal composition for the treatment of liver injuries and a process of preparing the herbal composition.

### BACKGROUND AND PRIOR ART REFERENCES

Eastern countries have an abundant source of ancient traditional medicine methodologies and practices. These methods may be, in part, responsible for the increased life expectancy of populations in eastern societies, compared to that of small island nations and western countries. In fact, most people contribute this high life expectancy to diet and exercise, amongst a plethora of other factors. Interestingly enough, most traditional medicine techniques include dietary principles and physical practices (e.g. cleansing diets, tea decoctions taken regularly, yoga, etc.). Unfortunately, in the small island chain of Micronesia, traditional ways have given way to western influence, including, but not limited to, diet and western medicine. There is a need to bridge the gap between traditional and western medicines, but the question is, “How do we prove to a society that ‘taking a step back’ may in fact benefit overall health? Is progress, by definition, moving forward to new and improved methods such as ‘pills and studied doctors’?” The answer is to develop medically useable methods of traditional/folk medicine, which are then validated by scientific research. This is a forward progression of traditional methods and will help to bridge the gap between medicine and culture. In most parts of Asia, there are already fully developed forms of Complementary and Alternative Medicine, where a positive basis for the study can be adapted and/or learned. Increased research and focus on Natural Medicines have sparked interest in, as well as validated, many traditional medicinal practices around the world. To preserve traditional medicinal knowledge, and document and verify medicinal plant products, we performed biological activity assessment of Palauan traditional medicines.

Hepatitis C virus (HCV), the major etiological agent of the non-A non-B hepatitis, was identified at the molecular level at the end of the 1980s (Choo et al., 1989; Houghton, 1996). The WHO currently estimates that Hepatitis C has been compared to a “viral time bomb”. According to the WHO, Initiative for Viral Research (IVR) portfolio for 2007, viral cancer report on Hepatitis C, about 180 million people, some 3% of the world's population, are infected with hepatitis C virus (HCV), 130 million of whom are chronic HCV carriers at risk of developing liver cirrhosis and/or liver cancer. It is estimated that three to four million persons are newly infected each year, 70% of whom will develop chronic hepatitis. HCV is responsible for 50-76% of all liver cancer cases, and two-thirds of all liver transplants in the developed world. Acute infection by HCV is only seldom diagnosed because of the vague clinical manifestations. However, more than 50% of infected individuals develop a slowly progressive chronic disease characterized by liver fibrosis and relatively specific symptoms that, although often not life-threatening, have adverse effects on the quality of life (Kenny-Walsh, 2001). Spontaneous healing is rare once a chronic infection has been established. Cirrhosis develops in 15-20% of the infected individuals and is accompanied by severe complications, leading eventually to liver failure and occasionally hepatocellular carcinoma (Francesco et al 2002).

Currently, preliminary screening of viable anti-HCV products is done on enzymatic assays, and efforts to develop new anti-HCV agents initially focused on viral enzymes, namely the NS3-4A serine protease and the NS5B RdRp. Both enzymes were later shown by genetic means to be essential for viral replication, thus validating their choice as targets for therapeutic intervention (Kolykhalov et al., 2000; Lohmann et al., 1999). In a further attempt to generate studies on Palauan plants and their therapeutic value, plants showing hepatoprotective effects on carbon tetrachloride treated primary hepatocytes were tested for their HCV-protease inhibitory activity. All of the aforementioned plants with high hepatoprotectivity showed promising results, but Phaleria nisidai was selected as the candidate for bioactivity-guided fractionation and chromatographic separation.

Phaleria nisidai, or Ongael in the Palauan language was described as a Palauan panacea (Matsuda 2004). P. nisidai's original name, Ongael, is more commonly known as, “Delal a Kar”. The name means “Mother of Medicine” and because of its use in an array of Palauan folk medicines, could be called Palau's Panacea. With the combination of hepatoprotective activity, HCV-protease inhibitory activity, and its uses in Palauan folk medicine, P. nisidai was subjected to bioactivity-guided fractionation and chromatographic separation.

### Objects of the Present Invention

The main object of the present invention is to develop an herbal composition for the protection against and treatment of liver injuries.

Another main object of the present invention is to develop a process for the preparation of the herbal composition from the Palauan medicinal plants for the protection against and treatment of liver injuries.

Yet another object of the present invention is to develop a method of protecting against and treating liver injuries, using the herbal composition from the Palauan medicinal plants.

Still another object of the present invention is to develop a method of treating liver injuries, using the herbal composition from the Palauan medicinal plants by suppressing the liver-damaging virus.

Still another object of the present invention is to develop a method of treating liver injuries caused by the Hepatitis C virus, using the specific fatty acid compositions that suppress viral replication.

### SUMMARY OF THE PRESENT INVENTION

The present invention relates to herbal compositions comprising the botanicals from the Palauan medicinal plants. The compositions of the invention have demonstrated outstanding efficacy for the treatment and protection of patients with hepatic disorders.

Compositions of the present invention have also inhibited the activities of liver-damaging virus activities, such as HCV. The preferred compositions for viral inhibition contain the botanical ingredients comprising the fatty acids or the phenolic constituents from the Palauan medicinal plants.

### BRIEF DESCRIPTION OF THE DRAWINGS

FIG. 1 represents fractionation with subsequent HCV-protease inhibitory activity.

FIG. 2 represents biologically guided fractionation of CHCl3 fraction.

FIG. 3 represents a fractionation of 22 from FIG. 7 with accompanying HCV-protease inhibition percentages (10 μg/ml).

FIG. 4 represents cells directly after inoculation.

FIG. 5 represents cells after the first medium change.

FIG. 6 represents CCl4 treated cells post sample aspiration.

FIG. 7 represents normal cells post sample aspiration.

FIG. 8 represents the hepatoprotectivity of Palauan Plants.

FIG. 9 represents the percentage inhibition of fatty acids (FIG. 10) including positive control (pc)(Embelinxghazi et al 2000) and fatty acid extract from P. nisidai.

FIG. 10 represents HCV protease inhibition by fatty acids.

FIG. 11 represents the percent inhibition of varying lengths of monoenoic fatty chains.

FIG. 12 represents HCV-protease inhibitory activity of 6 Palauan plant extracts at 1 mg/ml concentration.

FIG. 13 represents plant names with source and extract numbers for plants utilized in hepato-protectivity assay.

### DETAILED DESCRIPTION OF THE PRESENT INVENTION

Accordingly, the present invention relates to an herbal composition for the treatment of metabolic syndromes in a subject in need thereof, said composition comprising Phaleria nisidai, optionally along with hypoglycemic agents, a process thereof and also, a method of metabolic syndromes.

In still another embodiment of the present invention, wherein the herbal composition is in extraction form and any derivative of this form.

In Palau, alcoholism and the unregulated use of xenobiotics are rampant and an increasingly large problem. It is well documented that xenobiotics and ethanol are metabolized mainly in the liver (Parkinson 1996). The excessive introduction of these products into the body increases the likelihood of hepatic damage through oxidative stress. In lieu of this, research into hepatoprotective agents derived from Palauan folk medicine seems a valuable endeavor.

In-vitro models are indispensable in the initial screening of remedial therapeutic products. Important advances in hepatocyte primary cell line culture have allowed for hepatoprotective models to be used with increased accuracy (Papeleu 2005). Despite these advances, there is no standardized, and perfected method for the isolation and cell culture of primary hepatocytes. To find hepatoprotective agents from crude extracts of select Palauan folk medicinal plants, we used primary hepatocytes in a carbon tetrachloride-induced cell injury assay.

### Plant Materials:

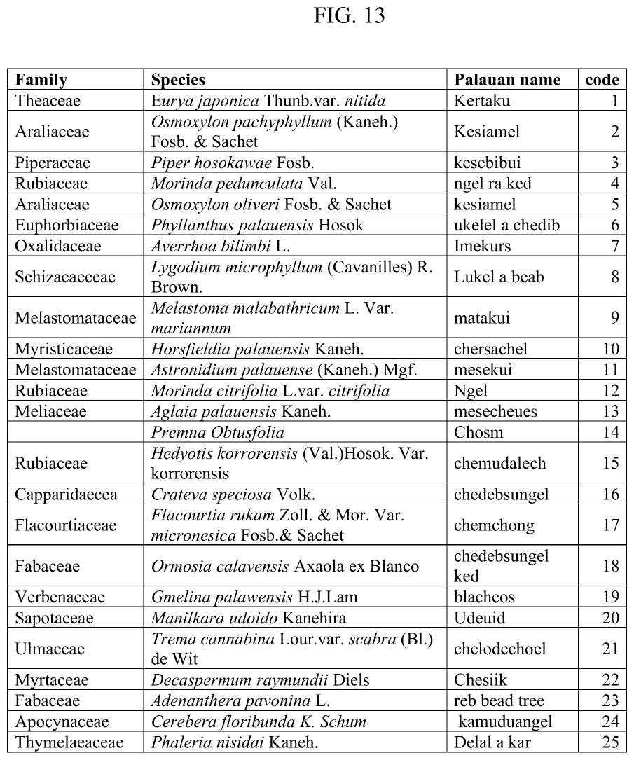

25 Palauan medicinal plants were screened for their hepatoprotective effects on carbon tetrachloride-treated primary hepatocytes (FIG. 13). Palauan plant leaves were collected and validated on-site by a Palauan botanist. Voucher samples are retained in the Belau National Museum Herbarium. Fresh leaves were dried under non-UV conditions and shipped, packaged in moisture absorbent casing, to Toyama University, Sugitani campus, after passing Palau Bureau of Agriculture Quarantine under research permit no: BOA04-05. Phaleria nisidai leaves were collected in bulk from three different sites on the Palauan islands and sites were marked using GPS coordinates. Plants were authenticated by local botanist Ann E. Kitalong on-site, and voucher specimens were stored in the Belau National Museum Herbarium. The bulk P. nisidai leaves were then air-dried under non-UV conditions and then placed in a light drier overnight. The leaves were then shipped, in moisture-absorbing packaging, to the University of Toyama, Sugitani Campus, after passing Palau Bureau of Agriculture Quarantine under research permit no: BOA04-05.

### Preparation of Extracts:

The plants were pre-weighed and extracted under reflux with 100 times the plants' dry weight in distilled water. Extracts were filtered through a cotton block and/or filter paper, then freeze-dried and stored. All samples were dissolved in DMSO at a concentration of 40 mg/ml, as stock solution. A stock solution was then diluted to 4 mg/ml with distilled water to minimize the DMSO effect on the cell-matrix.

### Hepatocyte Isolation:

### Procedure:

Following the basic methodology of a two-step collagenase perfusion (Seglan), oxygen saturated perfusion buffer was heated at 37° C. for 6-8 hours. Wistar rats were anesthetized with pentobarbital, and perfusion buffer was circulated through the portal cannula at approximately 50 ml/min. After the animal was fully anesthetized, a transverse incision was made at the lower abdomen, followed by a longitudinal incision along the linea alba to the sternum. Next, two transverse incisions were made from the superior edge of the longitudinal incision along the final rib until the abdomen could fold out to expose the internal abdomen and pelvic region. Clamps were used to reduce excessive bleeding. The gut and stomach were displaced to expose the vena cava, liver, and portal vein.

1,000 units of heparin were injected into the iliolumbar vein along the vena cava and a clamp was placed above the vein/injection site to inhibit bleeding. Directly proceeding with this procedure, a sterilized crescent-shaped needle and surgical thread were used to create a loose ligature around the portal vein, below the last tributary vein.

At this juncture, the peristaltic pump flow rate was adjusted to 20 ml/minute and shut off. Two mid-way cuts, in direct succession, were made into the lower vena cava-above the iliolumbar vein—and the portal vein, approximately 1 cm distal of the ligature. The portal cannula was inserted into the portal vein through the incision, pushed lcm past the ligature, and then the ligature was tightened enough to secure the portal cannula, and the peristaltic pump was switched on at a 20-ml/min-flow rate.

The liver was allowed to completely blanch, then two cuts were made completely through the vena cava, anterior and posterior to the liver. The liver was detached from the thoracic cavity and then connective tissue beginning with the biliary duct and proximate connective tissue was sectioned to facilitate liver detachment. Next, the portal vein distal of the portal cannula and proximate connective tissue (be careful not to displace the portal cannula during this process) were cut. Finally, the ligature was gripped with forceps and the liver was gently elevated and all remaining connective tissue was cut and liver was removed.

After completely removing the liver, it was placed onto a sturdy filter over a 500 ml receptacle, and the flow rate was increased to approximately 50 ml/minute. Perfusion was continued until fluid draining from the hepatectomized liver appeared clear. After approximately 20 minutes of initial perfusion, warmed collagenase dilute was transferred into a 100 ml reservoir, a peristaltic pump was stopped, tube end was transferred from perfusion dilute into the collagenase reservoir, the flow rate was decreased to 20 ml/min and the pump was switched back on. The cannula and liver were completely drained of perfusate, prior to transferring the liver to filter over the collagenase reservoir. The flow rate was increased to 30-40 ml/min for 15 to 20 minutes. The liver appearance was observed as partially granular and lighter in color-granular nature and lighter appearance indicate collagenase activity.

After perfusion with collagenase, the portal cannula was detached, the liver was submerged in warm perfusion buffer in a 50 ml reservoir, and the reservoir was placed in ice to gradually cool cells during the dissociation procedure. Expediently and gently the cells were disassociated from connective non-parenchymal cells with a blunt spatula in a sweeping motion from the periphery outwards.

Cells were then transferred into two 50 ml graduated centrifugation tubes and centrifuged at 0° C. at 20 g for two minutes. Then the supernatant was removed, and 35 ml of 0° C. Suspension Buffer dilute was added to each tube and gently shook to evenly disperse cell soup. The cell mixture was filtered through 100 μm filters, centrifuged at 30 g for three minutes, and the supernatant was removed. 35 ml of 0° C. Suspension Buffer dilute was added and cell soup was gently agitated and filtered through 100 μm. The filtered suspension was centrifuged at 30 g for 4 minutes and the supernatant was removed. 10 ml of medium was added, and the contents of both tubes were combined. Cell count and viability were measured by the Trypan-Blue exclusion method.

Hepatocytes were inoculated at 1×10³ cells, 200 μl volume, per well for 96-well collagen-coated plates (Carr 2007, Fukuda 2006) and at 2.5×10⁵ cells/ml, 3 ml volume, for 35 mm collagen-coated Petri dishes (Carr 2007, Fukuda 2006). The primary hepatocytes were then incubated at 37° C., 5% CO₂ for 2 (96-well plates) to 4 (35 mm Petri dishes) hours before the medium is changed and cells were monitored for adherence to the substrate. The medium was changed twice after 8 and 24 hours, for 96 well plates and 35 mm Petri dishes, respectively.

Directly proceeding with the second medium change, samples were introduced at 50 μg/ml and 200 μg/ml concentration and allowed to incubate at 37° C., 5% CO₂ for one hour. Then each respective well/dish was treated with 10 μM carbon tetrachloride (CCl₄)—long known to cause hepatic centrilobular necrosis and regarded as a classic hepatotoxin; its toxicity requires hepatic metabolism and involves free radical formation, lipid peroxidation, phosgene formation, and disturbances in Ca2+ homeostasis (Costa 1989)—and incubated for 8 (96-well plates) and 24 (35-mm dishes) hours 37° C. 5% CO₂. After incubation, the cell soup was centrifuged, and the supernatant was collected for analysis with LDH and GOT/GPT assay kits to ascertain the degree of cell injury compared to that of blank and negative controls.

### Assay Kits:

### Lactose Dehydrogenase:

The assay is based on the reduction of NAD by the action of LDH. The resulting reduced NAD (NADH) is utilized in the stoichiometric conversion of a tetrazolium dye. The resulting-colored compound is measured spectrophotometrically. If cell-free aliquots of medium from cultures given different treatments are assayed, then the amount of LDH activity can be used as an indicator of membrane integrity.

The supernatant of centrifuged cell soup after final incubation was collected in either 1.5 ml Eppendorf tubes or 96-well non-collagen coated well plates. LDH assay kit mixtures were combined directly preceding the assay, as indicated in the manual. 25 μl of well/dish supernatant were transferred to a fresh 96-well plate and 50 μl of LDH assay mixture were added in sequence. The assay was terminated 30 minutes after the addition of the LDH assay mixture by the addition of 10 μl of 1N hydrochloric acid. Spectrophotometric results for 96-well plates were read at 490 nm on a microplate reader.

### GOT/GPT:

Transaminases catalyze the transfer of an amino residue from an amino acid to an α-keto acid. Measurement of transaminase activity in serum is an important clinical test in the diagnosis of heart and liver diseases. The Transaminase C II kit (WAKO) utilizes a pyruvate oxidase method which uses N-ethyl-N-(2-hydroxy-3-sulfopropyl)-m-toluidine sodium salt and 4-amino antipyrine for the determination of GOT and GPT with almost no influence by coexisting substances.

The supernatant of centrifuged cell soup after final incubation was collected in 1.5 ml Eppendorf tubes. For 35 mm Petri dish samples, 0.5 ml GOT or GPT enzyme solution was added to 5 ml Falcon conical tubes and was incubated at 37° C. for 5 minutes. 5 μl of standard solution or cell supernatant was added to the 5 ml tubes and was incubated at 37° C. for 20 minutes at which point 2 ml of stop solution was added and samples were measured for absorbance at 555 nm on a UV spectrophotometer.

Data was collected and analyzed with by the following formula:

100×(Aₛₐₘₚₗₑ−Aₙₒᵣₘₐₗ)/(A_{negative}−Aₙₒᵣₘₐₗ)=% of CCl₄-induced cell death:

Where A is absorbance of sample, normal or negative control.

### Materials:

### Equipment Utilized:

A Kubota 6500 high-speed refrigerated centrifuge was used for pre-incubation centrifugation during isolation processes as well as for centrifugation of supernatant collected from 35 mm plates prior to enzyme analysis. A Beckman J6 Avanti™ J-series centrifuge was used to centrifuge 96-well plates prior to enzyme analysis. An InterMed ImmunoReader™ NJ-2100 UV plate reader was used for absorbance analysis of LDH assays at 490 nm. A Shimadzu UV-1650PC UV-visible spectrophotometric reader was used for absorbance analysis of GOT and GPT assays at 555 nm. Silicone tubing was routed through a peristaltic pump and one end was attached to tubing for portal vein cannulation. 500 ml Pyrex beaker was fitted with a filter paper support as a collection for perfusion dilute utilized during first step perfusion. 100 ml Pyrex beakers were used as reservoirs and filter paper was used as a placement for hepatectomized liver during both steps of the perfusion procedure. A 50 ml Pyrex beaker was used as a reservoir for initial placement and dissociation of hepatocytes from the perfused liver.

Buffer concentrates: All water/H₂O utilized was distilled and filtered through 0.25 μm to sterilize. Calcium concentrate: 4.5 g CaCl₂) to 2 molar H₂O, then filled up to 500 ml with water. Ca/Mg concentrate: 1.3 g MgCl₂ to 6 molar H₂O, then 1.8 g CaCl₂ and 2 molar H₂O, then filled up to 500 ml with water. Perfusion buffer concentrate: 207.5 g NaCl, 12.5 g KCl, 60.0 g HEPES, 6 g solid NaOH and then filled up to 1 liter with water. Suspension buffer concentrate: 40 g NaCl, 4 g KCl, 1.5 g KH₂PO₄, 1.0 g Na₂SO₄, 72 g HEPES, 69 g TES, 65 g Tricine, 21 g solid NaOH then filled up to 1 liter. Collagenase buffer concentrate was prepared in concession: container a; 1.25 collagenase type IV in 200 ml, added 1.75 g CaCl₂ in 2 molar concentration H₂O. In container b; 10 g NaCl, 1.25 g KCl, 60 g HEPES, and 6.6 g solid NaOH then filled up to 250 ml with water. Combine containers a and b and filled, if required 500 ml with water.

Buffer dilutes (saturated with Oz prior to use): Perfusion buffer dilutes: 20 ml of perfusion concentrate, filled up to 500 ml and heated to 37° C., and stored at 0° C. Collagenase buffer dilutes: Divided 10 ml of collagenase concentrate into individual 50 ml graduated cylinders and stored at −20° C. until used in isolation procedure. Immediately preceding the surgical procedure added 40 ml H₂O to collagenase concentrate and place it in a 37° C. water bath. Suspension buffer dilutes 20 ml suspension buffer concentrate, 10 ml Ca/Mg concentrate then added water up to 200 ml. Dilute was heated to 37° C. and stored at 0° C. (Seglan 1993).

Animals: Wister rats were kept in 5 rats in a cage, at 12-hour day/night cycles and were fed ad-lib.

Medium: Into William's E Medium or DMEM 6046 low glucose (Sigma) we added, 10% inactivated fetal bovine serum, epidermal growth factor at 0.01 mg/ml of medium (Carpenter 1990), insulin 0.5 mg/ml medium, dexamethasone 0.1 mM (Klein 2002) and 1% penicillin/streptomycin. The medium was heated to 37° C. and stored at 4° C.

Cell culture apparatus: collagen-coated 96-well and 35 mm Iwaki Petri dishes were utilized to increase hepatocytes' initial plating and proliferation.

### Biologically Guided Fractionation:

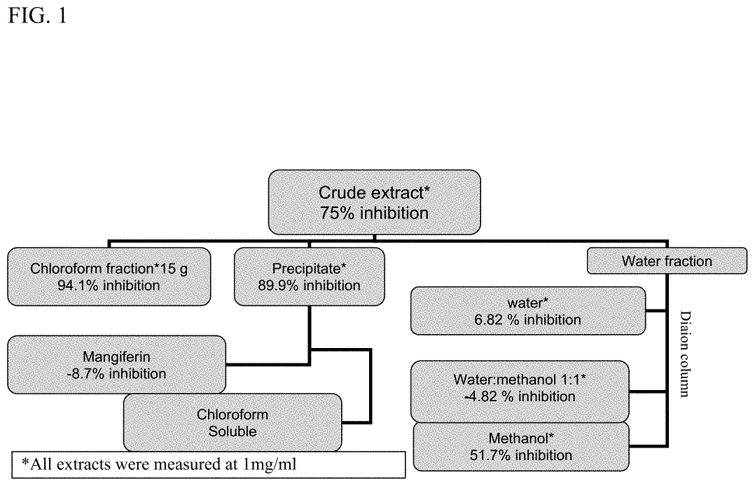

P. nisidai dried leaves were extracted with methanol, under reflux, for three hours, three times, and dried under reduced pressure. The crude extract was then dissolved in distilled water and mixed with chloroform to yield three fractions: a chloroform fraction, a precipitate fraction, and a water fraction. The water fraction was subsequently run over Diaion yielding three fractions: water, water; methanol 1:1, and methanol (FIG. 1).

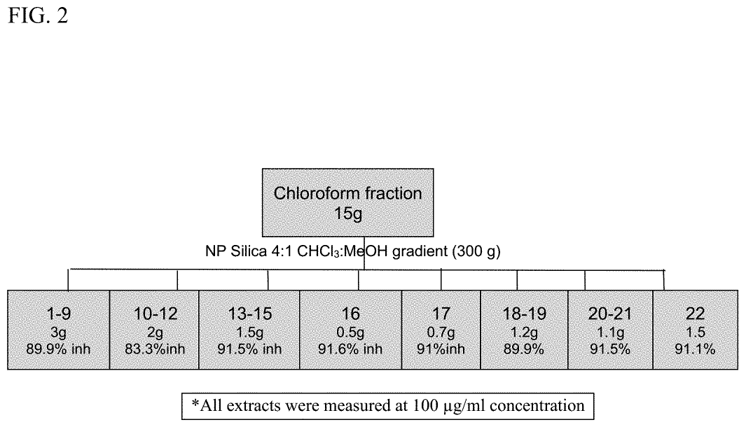

After determining HCV-protease activity, the chloroform fraction,—the most active fraction—was eluted over NP silica gel with chloroform and methanol at a 4 to 1 ratio, then increased by 10% methanol every 200 ml, yielding 22 fractions, combined according to TLC analysis of constituents. Fractions were tested for HCV-protease inhibition as shown in FIG. 2. Fraction twenty-two was for further isolation through TLC analysis-showing more RF variance in chemical constituents.

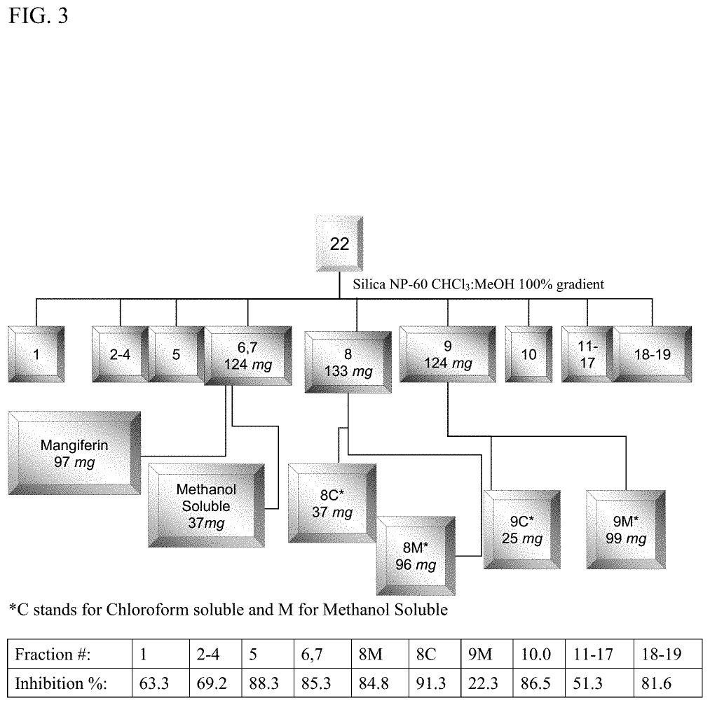

Fraction 22 was then run over NP-60 silica gel a second time with chloroform then a twenty percent increased gradient of MeOH every 100 ml. A small amount of precipitate formed in fractions 6-7, so it was dried and washed with MeOH to yield a mangiferin precipitate, and MeOH soluble fraction. The same procedure was carried out with fractions 8 and 9 but yielded no mangiferin, only a CHCl₃ soluble white, powdery substance, and a dark methanol eluent (FIG. 3).

Fraction 8C showed the highest activity and the powdery substance's NMR spectra revealed a possible mixture of fatty acids—with a characteristic fatty acid chain peak at δ1.219 and three sets of three triplet peaks from δ0.5-1.1.

Fatty acid identification with GC-MS: 1 mg of fatty acid mixture, 8C, was esterified by the addition of trimethylsilyl diazomethane (from TCI: Lot: QZKXB) to a methanol mixture of 8 C and allowed to react for 30 minutes under N₂ conditions with stirring. The reactant was then dried under reduced pressure and dissolved in 1 ml of CH₂Cl₂, then stored in a sealed container. Utilizing a Grain Fatty Acid Methyl Ester Mix, at 10 mg/ml concentration in methylene chloride (purchased from SUPELCO, Lot: LB43768), fatty acid standards were analyzed through GC-MS, on a Shimadzu GC-7A, JEOL auto-mass II spectrometer. GC conditions were as follows: column, DB-1, J & W Scientific, 0.25 mm×30 m; column temperature, 50-250° C. at 10° C./min, then 10 min at maximum temp; carrier gas was helium (Ma et al 1998). 2 μl each of standard fatty acid mix, esterified C8, and crude extract (esterified under the same conditions mentioned above; scaled up 10 times) were injected at separate times.

### HCV NS3/4a Protease Assay

### Principle of the Assay:

HCV NS3/4a protease cleaves viral non-structural polyprotein at four separate sites: NS3-4A, NS4A-NS4B, NS4B-NS5A, and NS5A-NS5B. SensoLyte™ 520 Protease Assay Kit *Flourimetric* implements a 5-FAM/QXL™ 520 FRET peptide whose sequence is derived from NS4A/NS4B cleavage site on the viral genome. The fluorescence of 5-FAM is quenched by the FRET peptide, and upon cleavage by the HCV NS3/4A protease, fluorescence can be measured at excitation/emission=490/520 nm. The competitive binding or direct inhibition of the samples is quantified by comparison vehicle, substrate, and test compound control, recorded by measuring fluorescence.

### Materials for HCV Protease Assay:

SensoLyte™ 520 Protease Assay Kit *Flourimetric* (lot #AK71145-1009) and HCV NS3/4A protease (lot #046-079) were purchased from AnaSpec, San Jose, Calif., USA, and components were utilized according to the manual with minor discrepancies. A BD Falcon™ Microtest™ 384-well 120 μl black Assay plate (lot #05391155) was utilized for the assay.

### Assay Procedure:

All samples were pre-weighed and dissolved in DMSO at varying concentrations. Crude extracts were applied to the assay at 1 mg/ml concentration, while fractions and compounds were assayed at 100 μg/ml and 10 μg/ml concentrations. Initially, 2 μl of the sample at the aforementioned concentrations was added to each well. Subsequently, 8 μl of a freshly diluted enzyme (1 μg/ml) was added to the wells containing the sample and the plate was gently agitated to aggregate contents. Finally, 10 μl of the freshly diluted substrate (Ac-Asp-Glu-Dap(QXL™ 520)-Glu-Glu-Abu-COO-Ala-Ser-Cys(5-FAMsp)-NH₂) was reacted with the mixture for 1 hour at 37° C. under sequential rotational shaking.

### Control Well Parameters:

Vehicle control—2 μl of DMSO in place of sample solution. Substrate control—10 μl buffer solution was mixed with 10 μl substrate dilute. Test compound control—2 μl of the sample was added to 18 μl of buffer solution. Inhibitor control—2 μl of Embelin at sample parameters (Ghazi et al 2000).

Fluorescence was measured by TECAN GENios plate reader at excitation/emission 485/530 nm, and % inhibition was calculated as follows:

100×(F_{vehicle}−Fₛₐₘₚₗₑ)/F_{vehicle}=% inhibition,

where F is the fluorescence value of vehicle control or of the sample minus the fluorescence of the substrate control. Compounds or extracts exhibiting fluorescence were measured and their values were subtracted from vehicle fluorescence.

### Results and Discussion:

Primary hepatocytes isolation proved to be an arduous task, and many parameters were altered during the course of the procedure, to improve reproducibility and accuracy. The limiting factor in isolation and adherence of the hepatocytes was time and oxygen saturation. The 2-step collagenase perfusion and cell isolation procedure (Seglen 1993) was truncated, so as to ensure cell survival. Cell adherence to collagen was improved by oxygen saturation of perfusion buffers directly prior to use.

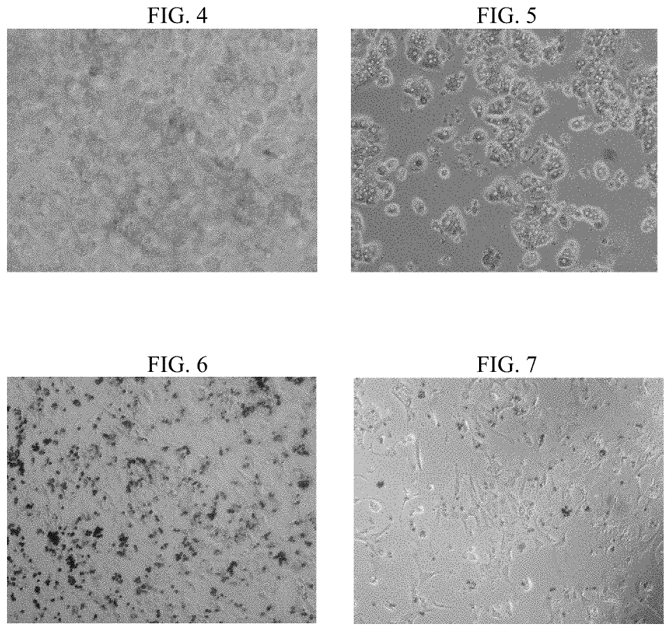

After initial inoculation, cells maintained their rounded shapes and had not adhered fully to the collagen surface (FIG. 4). After one hour and a medium change, the adherent cells had already initiated cell differentiation and division (FIG. 5). It is important to note the elongated characteristics and the obvious mitotic cleavage lines in the cells, as opposed to a number of nuclei—hepatocytes are multi-nuclei cells.

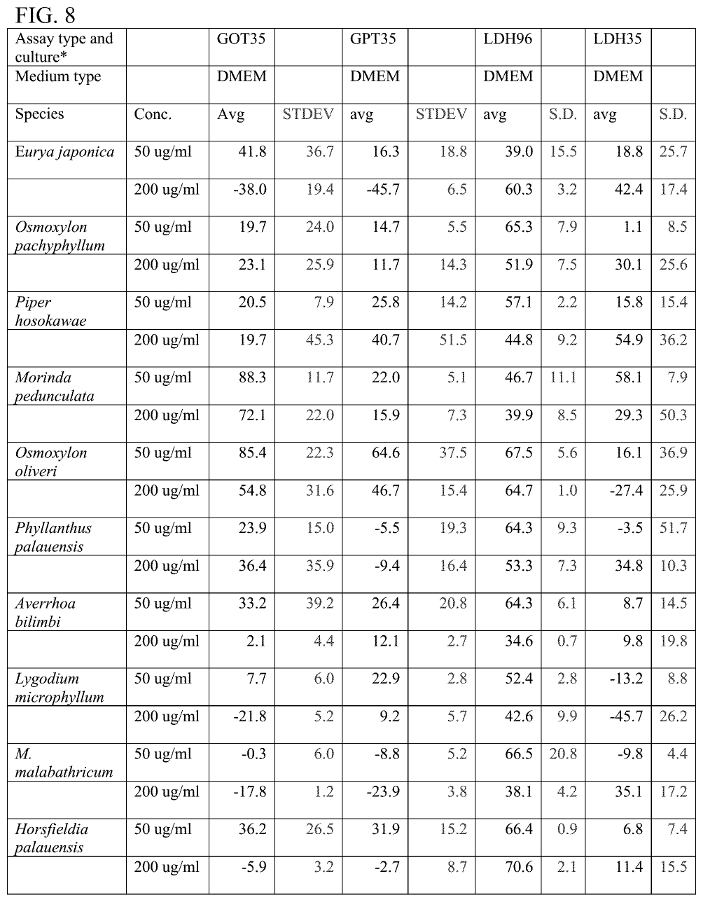

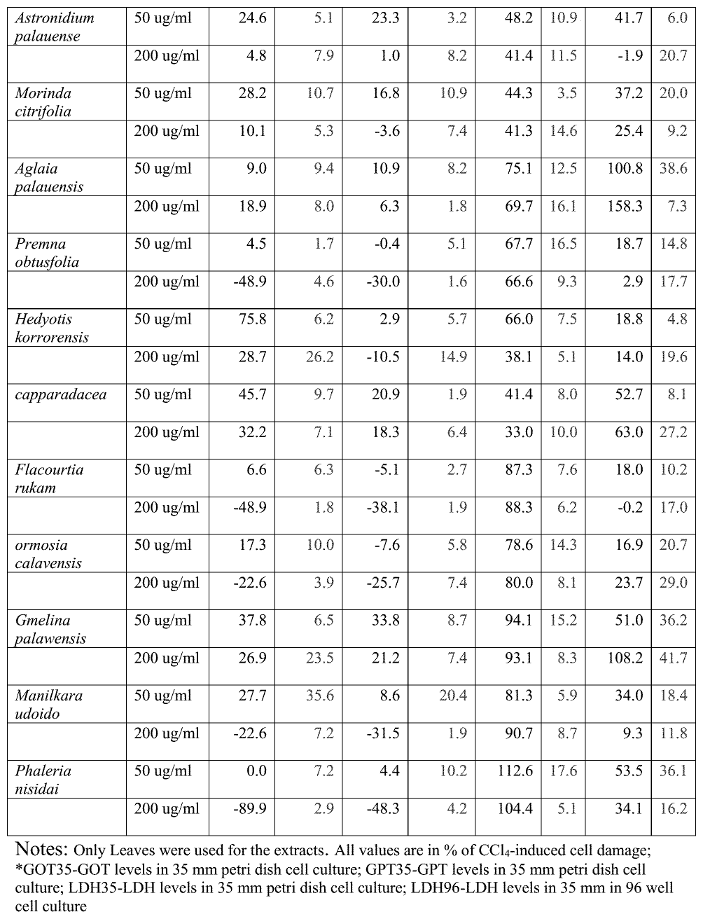

Necrotic cells were visible in all plates/dishes as evidenced by the dark cell bodies in the CCl₄-treated cells and normal cells (FIGS. 6 and 7, respectively). The degree of cell death in the sample treated cell cultures was, therefore, relative to the difference in necrotic factors between the carbon tetrachloride treated cell culture and normal cell culture, as prefaced in the calculation methods for percent reduction of CCl₄-induced cell death. Results were varying and the degree of deviation was marginal, due to less predictability of primary cell lines. FIG. 8 gives the hepatoprotectivity of plant extracts; each sample was triplicated, and the average values are shown.

Astronidium palauense, Averrhoa bilimbi, Flacourtia rukam, Gmelina palawensis, Hedyotis korrorensis, Lygodium microphyllum, Manilkara udoido, Morinda pedunculata, Osmoxylon oliveri, Phaleria nisidai and, Premna obtusfolia showed relatively high hepatoprotectivity in a dose-dependent matter. Palauan people typically use the plant leaves in decoctions boiled repeatedly, therefore the results were indicative of the hepatoprotective effects of folk medicines. These results may act as parameters for further investigation into the effects of Palauan folk medicine on other liver-related ailments resulting from hepatic damage.

C8 showed three major peaks at (written from largest peak to smallest peak):

peak 1: Rₜ=17.11 min, m/z; 270, 239, 227, 199, 185, 171, 157, 143, 129, 97, 87, 74, 55.
peak 2: Rₜ=19.11 min, m/z; 298, 281, 255, 199, 185, 171, 157, 143, 129, 97, 87, 74, 55
peak 3: Rₜ=23.49 min, m/z; 355, 341, 281, 221, 207, 149, 133, 117, 96, 82, 57

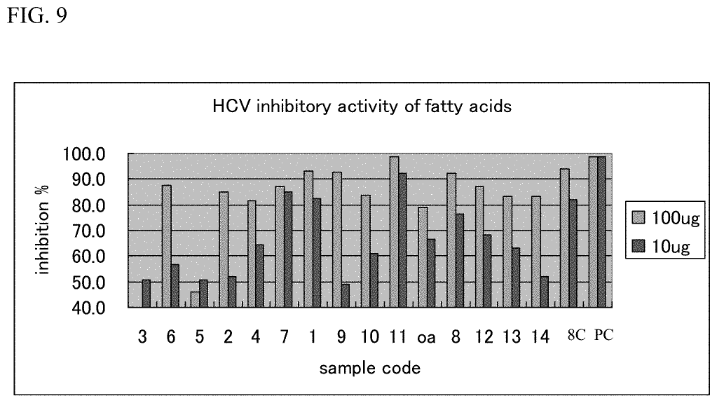

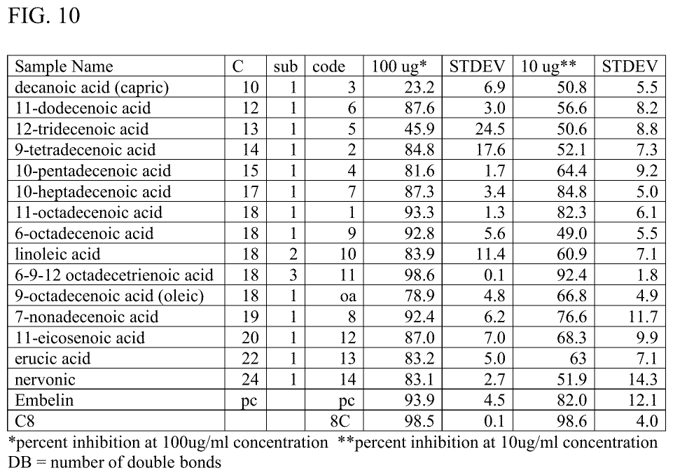

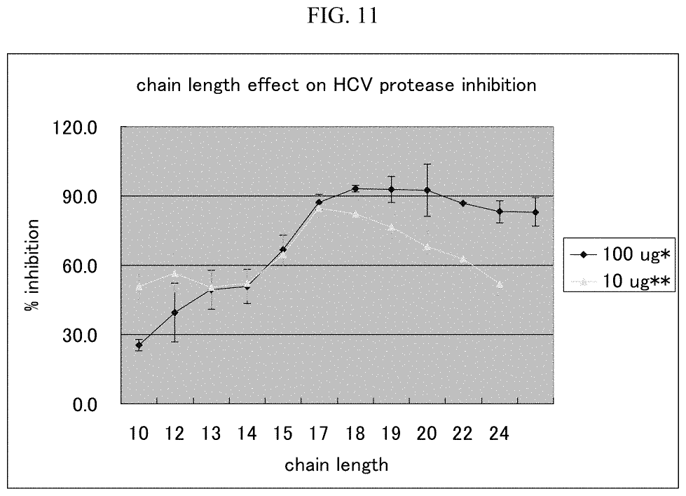

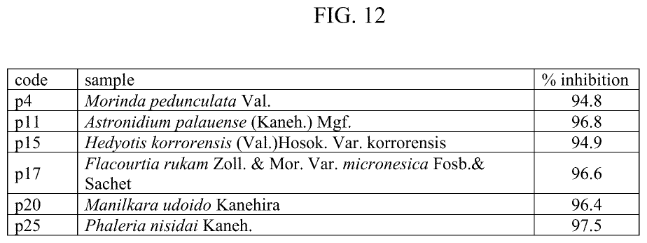

By comparison to standard samples, FA 1 was determined as palmitic acid, FA 2 was determined as stearic acid and FA 3, is suggested as behenic acid. To determine a base line for fatty acid inhibition of HCV-protease activity, different chain length, un-saturated and saturated fatty acids were assayed. The fatty acids tested seem to show activity with respect to carbon chain length. Graphing the result of monoenoic fatty acids at both 100 μg/ml and 10 μg/ml concentration revealed that a maximum inhibition at 18 carbon length chain. FIG. 9 shows that, select inhibition peaked at 18 carbon lengths and then decreased upon increased carbon chain.

Fatty acids' anti-HCV activity (Leu et at 2007) and HCV RNA replication inhibition has been documented. (Yano et al 2007). This study shows the HCV-protease inhibition of fatty acids and determines rate-limiting variations of fatty acids which may provide further insight into the therapeutic actions of fatty acids. In addition, this study shows the high activity of fatty acid extracts from P. nisidai, and segues into the additional study of its other chemical constituents.

### CONCLUSION

There is a dire need to maintain health and well-being, whether it be in large developed countries or small islands in the Pacific. Advances in science have cast a shadow over traditional methods of healing and have left smaller societies at a standstill in the advancement of healthcare. In an attempt to close this gap, this study was carried out in order to initiate the development of medicinal products from natural sources in Palau and promote healthcare through the validation and use of folk medicine.

A proper essay for initial studies was decided by an assessment of the health problems of Palau, as well as global health needs. The hepatoprotective screening was chosen as a viable screen because of lifestyle diseases, related to alcoholism and improper use of xenobiotics on the islands. The two aforementioned toxins are activated and/or cause degradation of the liver through a variety of pathways. In order to recreate this malady in-vitro, primary cell cultures were treated with our natural product extracts and were then exposed to a toxin (CCl₄) to mimic the effects of liver injury. Our results showed the hepatoprotective of the leaves of Astronidium palauense, Averrhoa bilimbi, Flacourtia rukam, Gmelina palawensis, Hedyotis korrorensis, Lygodium microphyllum, Manilkara udoido, Morinda pedunculata, Osmoxylon oliveri, Phaleria nisidai, Premna obtusfolia, providing an important milestone in the development of Palauan medicinal plants, like teas and/or elixirs, for hepatoprotective. In addition, these results acted as a basis for further analysis into other liver-damaging ailments, such as inhibition of liver-damaging viruses.

Chronic HCV, a liver-damaging virus, is responsible for 50-76% of all liver cancer cases, and two-thirds of all liver transplants in the developed world, it seemed ideal to screen Palauan plants that showed hepatoprotective for anti-HCV activity. Preliminary results of dual actions from therapeutic sources, such as Palauan medicinal plants, may provide insight into not only specific agents responsible for biological activity, but an overall remedial product that can be utilized daily or to supplement treatment. P. nisidai showed high activity in both studies, warranting further study into its causative components.

The biologically guided fractionation of the leaves of P. nisidai led to the conclusion that the fatty acid portion had the highest anti-HCV protease activity. The identification and authentication of these results were done via the development of a fatty acid key, for the chain length of monoenoic fatty acids versus inhibitory activity. An eighteen-carbon fatty acid chain appeared to be the best inhibitor of HCV-protease activity. This finding provides further insight into not only the remedial effects of fatty acids but on their availability in the Palauan plant. This, according to the best of our knowledge, is the first report of fatty acids' inhibitory activity on HCV-protease activity.

In addition to fatty acid isolation, the phenolic constituents of P. nisidai were determined. Three compounds, one of which was new and was determined as 2,4,6,4′-tetrahydroxybenzophenone 2-O-α-L-rhamnopyranoside, were isolated from the dried leaves of P. nisidai, and other phenolic derivatives were analyzed by LC-MS and MS². The phenolic content of P. nisidai, may be one of the reasons for its therapeutic efficacy in Palauan folk medicine.

The integration of Complementary and Alternative Medicine (CAM) and western medicine, is an important facet of this research, to develop an ideal means of providing healthcare in small nations that have more limited access to western medicine. The validation of folk medicine is a step in increasing medicinal repertoire, and therefore increasing health.

These are novel works and the patents based on them shall be used to increase focus on traditional medicines and promote their use to prevent and treat disease. Furthermore, the development of treatments from these patents will help stimulate a proper and sustainable economy for small islands through cultural and scientific means.

### REFERENCES

Agrawal, P. K., and Bansal, M. C. (1989). In Carbon-13 NMR of flavonoids, ed. by P. K. Agrawal, Elsevier, Amsterdam.
Breitmayer, E., Voeter, W., 1989. Carbon-13 NMR spectroscopy. VCH, Weinheim. Carpenter, G., Cohen, S. Epidermal growth factor J. Biol. Chem. Vol. 265, No. 14, pp. 7709-7712. 1990
Carr, I. B., Kar, S., Wang, M., Wang, Z. Growth inhibitory actions of prothrombin on normal hepatocytes: Influence of matrix. Cell Biol. Internat. 929-938. 2007
Choo, Q.-L., Kuo, G., Weiner, A. J., Bradley, L. R. D. W., and Houghton, M. Isolation of a cDNA clone derived from a blood-borne non-A non-B viral hepatitis genome. Science 244, 359-362. 1989
Costa, K. A., Trudell, R. J. Interaction of Hypoxia and Carbon Tetrachloride Toxicity in Hepatocyte Monolayers. Exp. and Molec. Path. 50, 183-192. 1989
De Francesco, R., Tomei, L., Altamura, S., Summa, V., Migliaccio, G. Approaching a new era for hepatitis C virus therapy: inhibitors of the NS3-4A serine protease and the NS5B RNA-dependent RNA polymerase Antiviral Res. 58 1-16. 2003
Defilipps, R. A., Maina, S. L., Pray, L. A. The Palauan and Yap Medicinal Plant Studies of Mayayoshi Okabe, 1941-1943 ATOLL RESEARCH BULLETIN No. 317, issued by National Museum of Natural History, Smithsonian Institution. 1988
Fukuda, J., Sakai, Y., Nakazawa, K. Novel hepatocyte culture system developed using microfabrication and collagen/polyethylene glycol microcontact printing. Biomaterials 27, 1061-1070. 2006
G. Michalopoulos, H. D. Cianciulli, A. R. Novotny, A. D. Kligerman, S. C. Strom, and R. L. Jirtle. Liver Regeneration Studies with Rat Hepatocytes in Primary Culture. Cancer Res. 42, 4673-4682. 1982
Hussein, G., Miyashiro, H., N., Hattori, M., Kakiuchi, N., Shimotohno, K. Inhibitory effects of Sudanese medicinal plant extracts on Hepatitis C virus (HCV) Protease. Phytother. Res. 14, 510-516. 2000
Kenny-Walsh, E. The natural history of hepatitis C virus infection. Clin. Liver Dis. 5, 969-977. 2001
Klein, H. H., Ullmann, S., Drenckhan, M., Grimmsmann, T., Kirsten Unthan-Fechner, Irmelin Probst. Differential modulation of insulin actions by dexamethasone: studies in primary cultures of adult rat hepatocytes. J. of Hepat. 37 432-440. 2002
Kolykhalov, A. A., Mihalik, K., Feinstone, S. M., Rice, C. M. Hepatitis C virus-encoded enzymatic activities and conserved RNA elements in the 3 nontranslated region are essential for virus replication in vivo. J. Virol. 74, 2046-2051. 2000
Leu, G., Lin, T., Hsu, T. J. Anti-HCV activities of selective polyunsaturated fatty acids. Biochem. Biophys. Res. Comm. 318 275-280. 2004
Lohmann, V., Korner, F., Herian, U., Bartenschlager, R. Biochemical properties of hepatitis C virus NS5B RNA-dependent RNA polymerase and identification of amino acid sequence motifs essential for enzymatic activity. J. Virol. 71, 8416-8428. 1997
Ma, C., Nakamura, N., Miyashiro, H., Hattori, M., Himotohno, K. Inhibitory effects of constituents from Cynomorium songaricum and related triterpene derivatives on HIV-1 protease. Chem. Pharm. Bull. 47 141-145. 1998
Markham, K. R., Wallace, J. W., Petone, N. Z. The chemotaxonomy of the Hymenophyllaceae. Part 1. C-glycosylxanthone and flavonoid variation within the filmy-ferns (Hymenophyllaceae). Phytochemistry, 19(3), 415-20. 1980
Matsuda, H., Tokunaga, M., Hirata, N., Iwahashi, H., Naruto, S., & Kubo, M. (2004). Studies on Palauan Medicinal Herbs. I. Antidiabetic Effects of Ongael, Leaves of Phaleria cumingli (Meisn.) F. Vill. Natural Medicines, 58(6), 278-283.
Murakami, T., Tanaka, N., Wada, H., Saiki, Y., Chen, C. M. Chemical and chemotaxonomical studies on filices. LXIII. Xanthone derivatives of Hypodematium fauriei Tagawa, H. crenatum Kuhn and Gymnocarpium robertianum Newm. (G. jessoense Koidz.). Yakugaku Zasshi 106(5) pp. 1986
Nott, E. P., Roberts, C. J. The Structure of mangiferin. Phytochemistry 6 741-747 1966
Papeleu, P., Vanhaecke, T., Henkens, T., Elaut, G., Vinken, M., Snykers, S., Rogiers, V. Isolation of Rat Hepatocytes. In: Methods in Molecular Biology vol. 320: Cytochrome P450 Protocols Second Edition ed. I. R. Phillips and E. A. Shephard C Humana Press Inc., Totowwa, N.J. 2005
Park, B. Y., Min, B. S., Oh, S. R., Kim, J. H., Bae, K. H., Lee, H. K. Isolation of flavonoids, a biscoumarin and an amide from the flower buds of Daphne genkwa and the evaluation of their anti-complement activity. Phytotherapy Research 20(7), 610-613. 2006
Parkinson, A. Biotransformation of xenobiotics. In: Klaassen C D, ed. Casarett and Doull's Toxicology. The Basic Science of Poisons, 5th edn. New York: McGraw Hill, 1996; 113-86.
Rancon, S., Chaboud, A., Darbour, N., Comte, G., Bayet, C., Simon, I. P., Raynaud, J., Pietro, D. A., Cabalion, P., Barron, D. Natural and synthetic benzophenones: interaction with the cytosolic binding domain of P-glycoprotein. Phytochemistry 57 553-557. 2001
Rosenberg, Steven. Recent advances in the molecular biology of Hepatitis C Virus. J. Mol. Biol. 313, 451-464 2001
Seglen, O. P. Isolation of Hepatocytes by Collagenase Perfusion, Methods in Toxicology. volume 1a, 231-243. 1993
Yano, M., Ikeda, M., Abe, K., Dansako, H., Ohkoshi, S., Aoyagi, Y., Kato, N. Comprehensive Analysis of the Effects of Ordinary Nutrients on Hepatitis C Virus Replication in Cell Culture. Antimicro. Agents and Chemother. 51. 2007

1. A method for isolating at least one fatty acid from a whole organic solvent plant extract of Phaleria nisidai, comprising steps of:
(a) providing the whole organic solvent plant extract of Phaleria nisidai containing at least one fatty acid amongst all other chemical compounds in the plant;
(b) partitioning with varying non-polar solvents with the extract containing at least one fatty acid amongst all other non-polar compounds in the plant;
(c) isolating with chromatographic separation of partition product with varying solvent concentrations over normal solid-phase silica gel allows for at least one fatty acid to be separated from other compounds into the specific fractions;
(d) assessing biological activity of these dried fractions, reconstituted in water allowing for validation of activity of at least one fraction from the extract with the possibility of containing one or more fatty acids amongst any other non-polar compound; and
(e) separating and identifying by using a Gas Chromatograph and detection with a tandem Mass Spectroscopy, respectively, providing chemical identification through a mass of at least one fatty acid amongst many other fatty acids and non-polar compounds.

2. The method according to claim 1, wherein the existence of at least one fatty acid is verified by the specific Gas Chromatography with the tandem Mass Spectrometry of (b1) esterified treatment of chloroform partition and multiple chromatographic column fractionations of methanolic whole extract (a1).

3. The method according to claim 1, further comprising multi-phase stepwise isolation by direct chromatographic separation of chloroform fraction over 300 grams of hydrocarbon 18 ‘normal’ solid phase silica gel with liquid phase eluant of a ratio of 4:1 CHCl₃: MeOH with a varying gradient to full CHCl₃, to yield 22 fractions; of which fraction 22 is run over slightly variant liquid phase conditions to yield 9 fractions.

4. The method according to claim 3, further comprising a step of drying fraction number 8 of 9 fractions and then washing fraction by multiple steps of resuspending in methanol; then removal through aspiration of methanolic soluble compounds leaving only chloroform soluble compounds containing at least one fatty acid amongst other non-polar compounds.

5. The method according to claim 4, further comprising a step of GC-MS on a specific silica column, with inert gas variations to reveal multiple fatty acids amongst other non-polar compounds.

6. The method according to claim 1, further comprising a step of running validation studies on varying fatty acids giving a relative biological activity of fatty acids, some of which are in the plant and others which are not.

7. The method according to claim 1, further comprising a step of running all separated fractions and compounds over an HCV NS1 protease inhibition immunological biological assay model for validation and guidance on step-wise fractionation for chemical separation and isolation of active fractions.

8. The method according to claim 1, further comprising a step of standardized development of screening an array of standardized fatty acids varying in chain lengths against biological activity yielding an ideal chain length of activity from 14-24 carbon monomeric fatty acids for biological activity.
